(19) United States
(12) Patent Application Publication
(10) Pub. No.: US 2024/0418724 A1
(43) Pub. Date: Dec. 19, 2024

(54) MOLECULAR MARKER FOR DETERMINING VERY-EARLY-STAGE ONSET RISK OF GASTRIC CANCER AND EVALUATING PROGRESSION RISK OF PRE-CANCEROUS LESION OF GASTRIC CANCER, AND USE THEREOF IN DIAGNOSTIC KIT

(71) Applicants: Shao LI, Beijing (CN); Peng ZHANG, Beijing (CN); TSINGHUA UNIVERSITY, Beijing (CN)

(72) Inventors: Shao LI, Beijing (CN); Peng ZHANG, Beijing (CN)

(21) Appl. No.: 18/704,212

(22) PCT Filed: Apr. 4, 2022
(86) PCT No.: PCT/CN2022/085212

§ 371 Date: Apr. 24, 2024

(30) Foreign Application Priority Data
Oct. 24, 2021 (CN) 202111236948.3

(51) Int. Cl. G01N 33/574

(52) U.S. Cl. CPC G01N 33/57446; G01N 2333/4742; G01N 2333/78; G01N 2333/96455

(57) ABSTRACT

Monoclonal antibodies of molecular markers (KRT7, KLK10 and LAMC2) for specifically marking very-early-stage cells of gastric cancer are prepared, and are used in the preparation of a kit for determining the very-early-stage onset risk of gastric cancer or other digestive tract tumors on the basis of gastric tissue or blood. The results show that: 1) the overall accuracy rate of predicting the progression risk of low-grade dysplasia reaches 86%, and the AUC value reaches 0.87; 2) the accuracy rate is closely related to the progression time of low-grade dysplasia, wherein the accuracy rate is increased to 95% 6 months before the onset of gastric cancer, and the window for the early diagnosis of gastric cancer can be moved forward by an average of 10 months; and 3) the accuracy rate of the marker in diagnosing gastric cancer is over 97%.

### CROSS-REFERENCE TO RELATED APPLICATIONS

This application is a national phase entry under 35 U.S.C § 371 of International Application No. PCT/CN2022/085212, filed on Apr. 4, 2022, which claims priority to Chinese Patent Application No. 202111236948.3, filed on Oct. 24, 2021, the entire content of all of which is incorporated herein by reference.

### REFERENCE TO AN ELECTRONIC SEQUENCE LISTING

The contents of the electronic sequence listing (File Name: 18704212.ST25.ASCII: Size: 13 kilobytes; and Date of Creation: Jun. 11, 2024) are herein incorporated by reference in its entirety.

### TECHNICAL FIELD

The present invention relates to a biomarker combination and measurement system for assisting in determining the risk of very early occurrence of gastric cancer and other gastrointestinal tumors, as well as evaluating the risk of progression of gastric precancerous lesions such as low-grade dysplasia.

### BACKGROUND TECHNOLOGY

Early diagnosis of gastric cancer is of great significance. Intestinal gastric cancer, as a major subtype of gastric cancer, undergoes a series of precancerous processes such as dysplasia and etc. Assessing the risk of progression from precancerous lesions to gastric cancer and achieving early diagnosis of gastric cancer are of great significance for the prevention and treatment of gastric cancer.

The duration of gastritis and cancer transformation is long, making it difficult to define the key transformation point, also known as the “starting point of cancer transformation”, which is a key difficulty in early diagnosis of gastric cancer. The inventors discovered a group of very early gastric cancer cells that began to appear in the low-grade dysplasia stage of the stomach and had a high risk of cancer transformation by establishing the first international single cell map of gastritis and cancer transformation, and used these cells as markers for diagnosing very early gastric cancer. Exploring the molecular characteristics of early gastric cancer cells and establishing a specific diagnostic system for identifying early gastric cancer cells is expected to accurately evaluate the risk of progression of gastric precancerous lesions such as dysplasia and achieve early diagnosis of gastric cancer.

The occurrence of tumors in other organs of the digestive system, such as the intestine, esophagus, and pancreas, also undergoes a series of precancerous lesions. Taking the molecular characteristics related to early gastric cancer cells as the starting point, it is expected to establish a common approach for early diagnosis of digestive system cancers, which is also of great significance for the prevention and treatment of digestive system tumors.

### SUMMARY OF THE INVENTION

The present invention provides use of a reagent for detecting the expression levels of molecular combination of early gastric cancer cell markers (including KLK10, KRT7, and LAMC2) in preparation for evaluating the risk of low-grade dysplasia progressing to gastric cancer and determining the risk of early gastric cancer occurrence. The combination includes KRT7, KLK10, and LAMC2. The present invention defines the cells marked by the molecular combination in the low-grade dysplasia stage as very early gastric cancer cells, and defines the stage in which very early gastric cancer cells appear as the very early gastric cancer stage. This stage is the critical point at which low-grade dysplasia and other precancerous lesions of gastric cancer transform into gastric cancer, which is the starting point of cancer transformation. By evaluating the expression level of the molecular markers in the extremely early stage of gastric cancer and quantifying the content of gastric cancer cells in the sample, it can serve as an important basis for evaluating the risk of low-grade dysplasia progressing to gastric cancer and for the diagnosis of extremely early gastric cancer.

In order to improve the specificity of marker molecules, the present invention further prepares relevant monoclonal antibodies, which can more specifically detect the expression of marker molecules from clinical samples. Based on the above-mentioned antibodies, the present invention has developed a kit for detecting and evaluating the content of gastric cancer early-stage cells from clinical tissue samples, and has developed a system for accurately determining the risk of gastric cancer early-stage occurrence. It is expected to provide effective basis for patients with digestive system tumors such as gastric cancer to take relevant treatment measures or decisions, with a promising clinical application prospect.

According to one aspect of the present invention, use of a reagent for detecting the expression level of combinations of biomarkers is provided for preparation of products for determining the diagnosis of very early gastric cancer, wherein the biomarker combinations' components include KLK10, KRT7, and LAMC2. The combinations include: 1) KLK10 and LAMC2; 2) KRT7 and LAMC2; 3) KRT7, KLK10, and LAMC2.

According to another aspect of the present invention, the provided reagents include self-prepared KLK10 and LAMC2 monoclonal antibodies.

The site sequence of KLK10 monoclonal antibody is:

AEAALLPQNDTRLDPEAYGSPCARGSQPWQVSLFNGLSFHCAGVLVDQSW
VLTAAHCGNKPLWARVGDDHLLLLQGEQLRRTTRSVVHPKYHQGSGPILP
RRTDEHDLMLLKLARPVVLGPRVRALQLPYRCAQPGDQCQVAGWGTTAAR
RVKYNKGLTCSSITILSPKECEVFYPGVVTNNMICAGLDRGQDPCQSDSG
GPLVCDETLQGILSWGVYPCGSAQHPAVYTQICKYMSWINKVIRSN,

The site sequence of the monoclonal antibody against LAMC2 is:
>sp|Q13753|22-1193

TSRREVCDCNGKSRQCIFDRELHRQTGNGFRCLNCNDNTDGIHCEKCKNG
FYRHRERDRCLPCNCNSKGSLSARCDNSGRCSCKPGVTGARCDRCLPGFH
MLTDAGCTQDQRLLDSKCDCDPAGIAGPCDAGRCVCKPAVTGERCDRCRS
GYYNLDGGNPEGCTQCFCYGHSASCRSSAEYSVHKITSTFHQDVDGWKAV
QRNGSPAKLQWSQRHQDVFSSAQRLDPVYFVAPAKFLGNQQVSYGQSLSF
DYRVDRGGRHPSAHDVILEGAGLRITAPLMPLGKTLPCGLTKTYTFRLNE
HPSNNWSPQLSYFEYRRLLRNLTALRIRATYGEYSTGYIDNVTLISARPV
SGAPAPWVEQCICPVGYKGQFCQDCASGYKRDSARLGPFGTCIPCNCQGG
GACDPDTGDCYSGDENPDIECADCPIGFYNDPHDPRSCKPCPCHNGFSCS
VMPETEEVVCNNCPPGVTGARCELCADGYFGDPFGEHGPVRPCQPCQCNN
NVDPSASGNCDRLTGRCLKCIHNTAGIYCDQCKAGYFGDPLAPNPADKCR
ACNCNPMGSEPVGCRSDGTCVCKPGFGGPNCEHGAFSCPACYNQVKIQMD
QFMQQLQRMEALISKAQGGDGVVPDTELEGRMQQAEQALQDILRDAQISE
GASRSLGLQLAKVRSQENSYQSRLDDLKMTVERVRALGSQYQNRVRDTHR
LITQMQLSLAESEASLGNTNIPASDHYVGPNGFKSLAQEATRLAESHVES
ASNMEQLTRETEDYSKQALSLVRKALHEGVGSGSGSPDGAVVQGLVEKLE
KTKSLAQQLTREATQAEIEADRSYQHSLRLLDSVSRLQGVSDQSFQVEEA
KRIKQKADSLSSLVTRHMDEFKRTQKNLGNWKEEAQQLLQNGKSGREKSD
QLLSRANLAKSRAQEALSMGNATFYEVESILKNLREFDLQVDNRKAEAEE
AMKRLSYISQKVSDASDKTQQAERALGSAAADAQRAKNGAGEALEISSEI
EQEIGSLNLEANVTADGALAMEKGLASLKSEMREVEGELERKELEFDTNM
DAVQMVITEAQKVDTRAKNAGVTIQDTLNTLDGLLHLMDQPLSVDEEGLV
LLEQKLSRAKTQINSQLRPMMSELEERARQQRGHLHLLETSIDGILADVK
NLENIRDNLPPGCYNTQALEQQ.

According to another aspect of the present invention, an immunohistochemical detection kit is provided for immunostaining the protein expression levels of one or more combinations of the three protein molecules KLK10, LAMC2, and KRT7, to determine the content of gastric cancer cells in the very early stage and assist in the diagnosis of very early gastric cancer.

According to one aspect of the present invention, use of combinations of three molecules KLK10, LAMC2, and KRT7 is provided as features of the very early gastric cancer cell population and to determine the risk of low-grade dysplasia progressing to gastric cancer.

According to another aspect of the invention, use of the changing characteristics of combinations of three molecules KLK10, LAMC2 and KRT7 in pancreas, intestine, esophagus and other tissues from precancerous lesions to early cancers is provided to determine early pancreatic cancer, intestinal cancer and esophageal cancer.

According to another aspect of the present invention, it is provided to associate the expression levels of three molecules, KLK10, LAMC2, and KRT7 with the risk of postoperative recurrence and survival of gastric cancer, and to use this association for determining the risk of postoperative recurrence of gastric cancer.

According to another aspect of the present invention, a detection reagent kit is provided: the detection kit can be used for immunohistochemistry (IHC) staining of the above-mentioned protein molecules to obtain their expression in the tested gastric tissue, thereby determining the content of early stage gastric precancerous cells and early cancer cells in the tested tissue, and it can also be used for detecting the expression of the above-mentioned protein molecules in the blood through enzyme-linked immunosorbent assay (ELISA).

According to another aspect of the invention, a system and method for determining precancerous lesions of gastric cancer, early gastric cancer, early pancreatic cancer, early intestinal cancer, early esophageal cancer and/or determining postoperative recurrence risk of gastric cancer are provided. The system determines the number of gastric precancerous lesions and early cancer cells in gastric tissue based on the expression of cell marker proteins in tissues or blood, thereby determining the risk of developing gastric precancerous lesions and/or gastric cancer in the tested patient, and/or determining the risk of postoperative recurrence of gastric cancer in the tested patients. The system also determines the risk of other tumors of the digestive system, such as colon cancer, esophageal cancer, and pancreatic cancer, according to the expression of cell marker proteins in the tissues or blood of the tested patient.

The above-mentioned system according to one embodiment of the present invention comprises:
detection kit for immunohistochemical staining of the expression levels of very early gastric cancer cell markers KLK10, LAMC2, and KRT7 in the tested tissue, and/or for obtaining the content of very early gastric cancer cell markers in the blood samples of the tested patients through enzyme-linked immunosorbent assay:
very early gastric cancer cell counting and classifying device for determining the proportion and grade of KLK10, LAMC2, and KRT7 positive cells in the tested tissue samples, and/or for determining the overall expression levels of KLK10, LAMC2, and KRT7 in the serum of the tested blood samples by calculating the mean value.

According to a further aspect of the present invention, the above system performs classifying processing of expression levels based on the proportion of very early gastric cancer cells to the total number of cells in the entire test tissue, including negative (proportion=0%, Grade 0), low Grade (proportion<5%, 1), moderate (5%<proportion<30%, Grade 2), and high (proportion>30%, Grade 3), in order to determine the risk index for patients to progress to gastric cancer and/or the risk index for postoperative recurrence of gastric cancer; and/or for determining the risk index of esophageal cancer, pancreatic cancer and intestinal cancer according to the expression level of very early gastric cancer cell marker molecules in esophageal, pancreatic and intestinal tissues by using the above layering treatment method; and/or for determining the risk index of digestive system tumors of the tested patient using the overall expression level of marker molecules of gastric cancer cells in the serum.

According to another aspect of the present invention, use of a reagent is provided for determining the expression levels of three very early gastric cancer cell markers KLK10, LAMC2, and KRT7 to obtain their expression in the tested tissue and/or blood for the preparation of a composition for determining early gastric precancerous lesions and/or early gastric cancer and other early digestive system tumors.

### EXPLANATION OF DRAWINGS

FIG. 1 is a schematic diagram of the system for gastric cancer precancerous lesion progression risk assessment and very early gastric cancer occurrence risk determination based on combinations of molecular marker monoclonal antibodies according to an embodiment of the present invention.

FIG. 2 is a box plot of the expression of biomarker molecules based on the combinations of very early gastric cancer cell markers according to an embodiment of the present invention, representing the expression of biomarkers in different outcome patient groups (low-grade dysplasia patients progressing to gastric cancer group, referred to as progression group: low-grade dysplasia regressing to non low-grade dysplasia group, referred to as regression group: low-grade dysplasia maintenance group, referred to as maintenance group)* Indicates p-value≤0.05.

FIG. 3 is the ROC curve diagram of using the very early gastric cancer cell marker molecule combination used to distinguish patients in the progression group and regression group according to the present invention.

FIG. 4A shows the overall predictive performance of the molecular combination of very early gastric cancer cell markers in relation to the time window of progression from low-grade dysplasia to gastric cancer. FIG. 4B shows the relationship between the predictive performance and progression time of the molecular combination of very early gastric cancer cell markers in Helicobacter pylori positive and negative cases, grouped by Helicobacter pylori infection.

FIG. 5 shows the correlation between the molecular combination of early gastric cancer cell markers, clinical pathological parameters, and the risk of progression from low-grade dysplasia to gastric cancer using the COX proportional risk model.

### DETAILED DESCRIPTION OF THE INVENTION

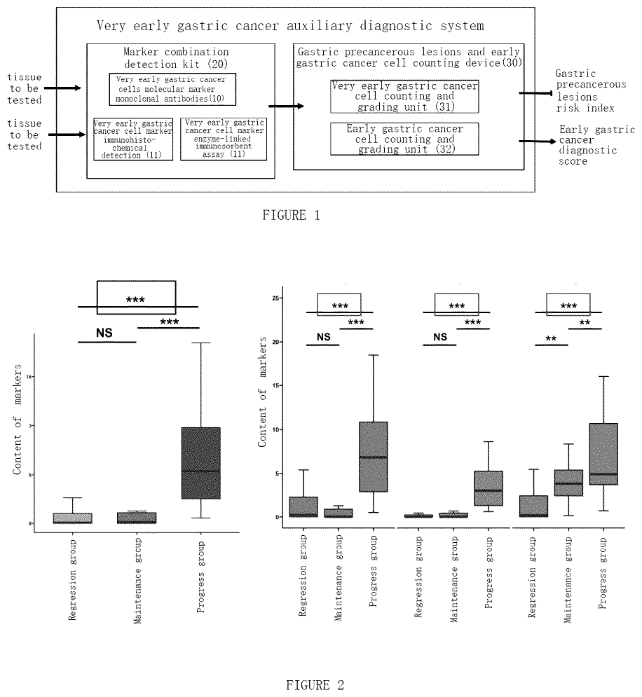

FIG. 1 is a schematic diagram of a gastric cancer risk assessment and very early diagnosis system based on a combination of very early gastric cancer cell markers according to an embodiment of the present invention.

Specifically, according to the first aspect of the present invention, a detection kit based on three molecules is provided. This kit includes monoclonal antibody reagents for detecting the expression levels of KLK10, LAMC2, and KRT7 molecules, as well as blank solution, dilution solution, and antigen repair solution. In a preferred embodiment of the present invention, the monoclonal antibody reagent is an antibody for immunohistochemical detection method, as shown in Table 1. According to another embodiment of the present invention, the monoclonal antibody reagent can also be used as an antibody for Western Blot or ELISA detection methods.

TABLE 1
List of Immunohistochemical Detection Antibodies Used for
the Tested Molecules and Preparation Concentration Ratios

| | | Biomarker | Preparation |
|---|---|---|---|
| Reagent number | antibody | molecule | concentration ratio |
| Reagent 1 | Anti-KLK10 | KLK10 | 1:200 |
| | antibody | | |
| Reagent 10 | Anti-Cytokeratin 7 | KRT7 | 1:200 |
| | antibody (ab53707) | | |
| Reagent 3 | Anti-LAMC2 | LAMC2 | 1:50 |
| | antibody | | |

According to the second aspect of the present invention, an immunohistochemical detection method is provided for the expression levels of the three molecules KLK10, LAMC2, and KRT7 in gastric tissue. In one embodiment of the detection method according to the present invention, in addition to the above-mentioned reagents contained in the reagent kit, the user may prepare or purchase the following reagents themselves:
(1) distilled water or de-ionized water:
(2) 3% H₂O₂;
(3) xylene:
(4) 75%, 85%, 95% alcohol and anhydrous ethanol;
(5) 10 mM TBS solution (pH 7.2-7.4): 1.21 g of trihydroxyaminomethane, 7.6 g of sodium chloride, adding 800 mL of distilled water, adjusting the pH value to 7.2-7.4 with concentrated hydrochloric acid, and finally make up to 1000 mL;
(6) 10 mM pH 6.0 citric acid buffer: 0.38 g citric acid, 2.45 g trisodium citrate, add 900 mL of distilled water, adjust the pH to 6.0 with concentrated hydrochloric acid, and finally fixing the volume to 1000 mL;
(7) hematoxylin solution;
(8) neutral resin.

### EMBODIMENTS

In order to verify the value of the present invention in assisting in the assessment of the risk of gastric cancer in patients with low-grade dysplasia of the stomach, the diagnosis of digestive system tumors such as very early gastric cancer, and the prediction of the risk of gastric cancer recurrence, the present invention conducts multi validation on multicenter and retrospective sequential cases collected clinically.

### Application of Combinations of Very Early Gastric Cancer Cell Markers in Determining the Risk of Very Early Gastric Cancer Occurrence

### Embodiment 1

### Biomarker Identification of Patient Population Inclusion and Analysis of and Clinical Characteristics

Firstly, the inventor retrospectively screened a total of 324 patients with low-grade dysplasia in a sequential cohort from three independent medical centers: Peking Union Medical College Hospital, Wangjing Hospital of the Chinese Academy of Traditional Chinese Medicine, and Yijishan Hospital in Anhui Province. The criteria for patient selection and inclusion are: 1) having two or more records of gastroscopy examinations: 2) the baseline diagnosis is low-grade intraepithelial neoplasia: 3) the patient did not suffer from accompanying diseases such as gastric cancer, gastric ulcer, or other tumors. Based on the final endoscopic diagnosis of the patient, they were divided into three groups: progressive (endpoint pathology is gastric cancer or high-grade intraepithelial neoplasia), maintenance (endpoint pathology is low-grade intraepithelial neoplasia), and regression (endpoint pathology is intestinal metaplasia or atrophic gastritis, etc.). Among them, there were 107 cases in the progressive group, 41 cases in the maintenance group, and 175 cases in the regression group, with an average follow-up monitoring time of 18 months (3-80 months).

By analyzing the baseline clinical characteristics of each group of patients, it was found that: 1) the average age of the enrolled patients was 59 years old, with a male to female ratio of 1.59, a proportion of Helicobacter pylori infection patients was 0.28, an average of 2.2 pathological records, and there was no significant correlation among various clinical indicators: 2) in each group, the average age of the progression group, maintenance group, and regression group was 63 years, 55 years, and 56 years, respectively: the male to female ratio was 2.29, 1.92, and 1.24, respectively: the proportion of patients infected with Helicobacter pylori was 0.21, 0.27, and 0.33: the average tracking time was 15.7 months, 15.9 months, and 18.9 months. In each center, the number of cases in Center 1, Center 2, and Center 3 was 110, 114, and 100, respectively: the average age was 53 years old, 56 years old, and 63 years old, respectively: the male to female ratio was 2.24, 1.71, and 1.04, respectively: the proportion of patients infected with Helicobacter pylori was 0.33, 0.21, and 0.32; the average tracking time was 17.2 months, 21 months, and 16.5 months.

### Detection of Expression of Very Early Gastric Cancer Combination Biomarkers

Firstly, the immunohistochemical detection kit (20) according to the present invention was used to obtain the expression of combined biomarkers based on three protein molecules KLK10, LAMC2, and KRT7 in the 324 pathological specimens. This kit uses immunohistochemistry (IHC) to measure the expression level of the combination markers.

The paraffin embedded surgical samples were fixed with 10% formalin buffer, and were sliced into 4 microns per piece.

The reagent kit in this embodiment includes:
(1) Reagent A: blocking solution, which was 10% goat serum:
(2) Reagent B: diluted ready-to-use anti-KLK10 primary antibody:
(3) Reagent C: diluted ready-to-use anti-LAMC2 primary antibody:
(4) Reagent D: diluted ready-to-use anti-KRT7 primary antibody:
(4) Reagent G: anti goat biotinylated secondary antibody:
(5) Reagent H: HRP labeled with streptavidin:
(6) Reagent I: 20× concentrated DAB substrate solution:
(7) Reagent J: 20× concentrated DAB substrate buffer solution:
(8) Reagent K: 20× concentrated DAB colorimetric solution.

In addition to the above-mentioned reagents included in the reagent kit, the following reagents were also self-prepared:
(1) distilled water or deionized water:
(2) 3% H₂O₂:
(3) Xylene:
(4) 75%, 85%, and 95% alcohol and anhydrous ethanol:
(5) 10 mM TBS solution (pH 7.2-7.4): 1.21 g of trihydroxyaminomethane, 7.6 g of sodium chloride, adding 800 mL of distilled water, adjusting the pH value to 7.2-7.4 with concentrated hydrochloric acid, and finally fixing the volume to 1000 mL:
(6) 10 mM pH 6.0 citric acid buffer: 0.38 g citric acid, 2.45 g trisodium citrate, adding 900 mL of distilled water, adjusting the pH to 6.0 with concentrated hydrochloric acid, and finally fixing the volume to 1000 mL;
(7) hematoxylin solution:
(8) neutral resin.

The above kit was used to detect the expression of combined markers in pancreatic cancer tissues:
(1) tissue embedding: the pancreatic cancer tissue samples were fixed with 10% neutral formalin for 2 h and were rinsed repeatedly with running water to remove the fixative, the sample were placed in 75% alcohol overnight, and then they were subjected to gradient dehydration with alcohol gradient: 75% alcohol for 1 h, 85% alcohol for 1 h, 95% alcohol for 1 h, and absolute alcohol for twice, 1.5 h each time: then they were immersed in xylene for 1.5 h, and immersed in wax for 1 h in an oven at 60° C., and were stored at 4° C. after cooling for standby:
(2) paraffin slicing: the wax blocks were trimmed and the slicing machine (SLEE paraffin slicer CUT5062) was adjusted to set the slice thickness to 3-4 μm: slicing was performed continuously, floating flattening was performed in 60° C. water, and laid flat on a glass slide coated with cationic resin:
(3) baking the slices: the slices to be made were placed on the slicing rack and baked in a constant temperature oven at 60° C. for at least 1 hour:
(4) dewaxing: the slices were put into a container containing xylene for dewaxing 3 times (i.e. xylene I, II, III), each time for 10 minutes:
(5) hydration: the slices were subjected to downward alcohol hydration, with anhydrous ethanol for 5 minutes, 95% ethanol twice (2 minutes each time), and 85% ethanol for 2 minutes: 75% ethanol for 2 minutes, and were rinsed with distilled water for 1 minute:
(6) antigen repair: 1000 ml of citric acid buffer were added to a pressure cooker, the slicing racks containing slices were immersed in the buffer and were repaired at high temperature and high pressure for 2 minutes and 45 seconds, and were washed with TBS 3 times, each time for 2 minutes:
(7) 3% H₂O₂ was added dropwise onto the slices and the slices were let stand at room temperature for 15 minutes, washed with TBS three times, for 2 minutes each time:
(8) sealing: reagent A was added dropwise onto the tissue slices, completely covering the tissue slices, and the tissue slices were incubated at room temperature for 10 minutes and then the liquid was dried without rinsing:
(9) adding primary antibody: different slices were added dropwise with reagent B (anti KLK10 primary antibody), reagent C (anti LAMC2 primary antibody), and reagent D (anti KRT7 primary antibody), respectively, completely covering the tissue slices: incubation were performed in a 37° C. wet box for 2 hours or overnight at 4° C.:
(10) washing: TBS-T washing (3×5 minutes):
(11) add secondary antibody: Reagent G (dropwise addition of biotinylated secondary antibody), which completely covered the tissue slices: incubation were performed in a 37° C. wet box for 30 minutes:
(12) washing: TBS washing 3 times, each time for 5 minutes:
(13) adding HRP-SA: dropwise addition of reagent H (HRP labeled with streptavidin) was performed, completely covering the tissue slices, and incubation were performed in a 37° C. wet box for 30 minutes:
(14) washing: TBS washing 3 times, each time for 5 minutes:
(15) preparation of DAB color solution: was prepared on site. For staining a slice, for example, 2.5 ul of reagent I was taken and added to 50 ul of distilled water and mixed well, then, 2.5 ul of reagent J and 2.5 ul of reagent K were added to the above liquid and mix well:
(16) color development: dropwise addition of the DAB color development solution mentioned above onto the tissue slices was performed, completely covering the tissue slices. Observation of color development under a microscope was performed and rinse with distilled water was performed to terminate the color development:
(17) re-staining: re-staining with hematoxylin for 3 minutes, differentiation with hydrochloric acid and alcohol:
(18) sealing: soak in 75% alcohol for 2 minutes, 85% alcohol for 2 minutes, 95% alcohol for 2 minutes, anhydrous ethanol for 2 minutes, then soak in xylene for 15 minutes, replacement with xylene was made and soak for another 15 minutes was performed, seal with neutral resin was performed:
(19) results interpretation: the stained pancreatic cancer tissue sections were observed under the microscope, and the positive results were stained with brownish yellow granules. Five high power fields (10*40) were randomly selected to count the number of positive cells. The proportion of positive cells at 0%, 1-5%, 6-30%, and 31-100% were determined as 0, 1, 2, and 3 points, respectively. The staining intensity of positive cells in each slice was determined as 0, 1, 2, and 3 points based on no staining, light yellow; brown yellow; and brown, respectively.

### Prediction of Cancer Risk for Low-Grade Gastric Dysplasia Using Combinations of Very Early Gastric Cancer Cell Markers

The inventors evaluated the cancer risk prediction of low-grade gastric dysplasia by very early gastric cancer cell markers from three aspects.

Firstly, the inventors evaluated the overall distribution of biomarker expression levels in three different groups. By quantifying the biomarkers into four stages: negative (0), weakly positive (1), positive (2), and strongly positive (3), the inventors found that the molecular expression levels of biomarkers in the progressive group were significantly higher than those in the regression group, regardless of the overall situation or individual centers (FIG. 2, p<0.01), indicating that the biomarkers have value in identifying the risk of low-grade dysplasia progression.

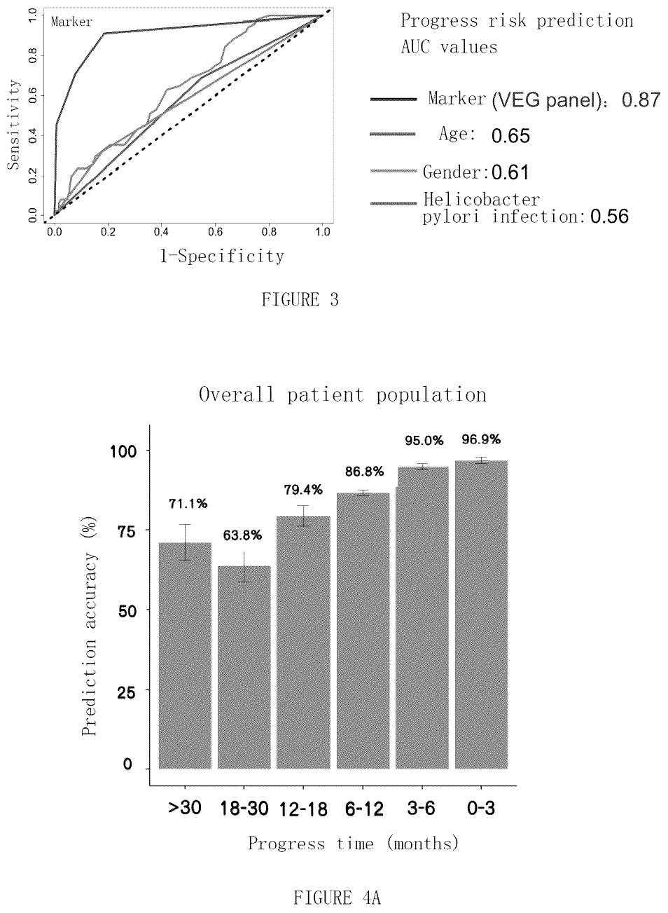

Furthermore, the inventors evaluated the predictive performance of the biomarkers in predicting the risk of low-grade dysplasia progression and in diagnosing very early gastric cancer. By plotting the ROC curves (FIG. 3) and comparing them with existing clinical pathological indicators (age, gender, Helicobacter pylori infection, etc.), it was determined that the predictive AUC value of the biomarkers for low-grade dysplasia progression risk (and very early gastric cancer) was 0.87, with an overall prediction accuracy of 84%. The above values are significantly better than existing clinical indicators such as patient age, gender, and H. pylori infection (p<0.001), further confirming the ability of the biomarker molecules to predict the risk of low-grade dysplasia and canceration and to determine the risk of very early onset of gastric cancer.

Furthermore, the inventor, utilized the COX proportional risk model, evaluated the association between biomarker expression and the risk of low-grade dysplasia progression, and assessed the coupling of this association with clinical pathological parameters. The results of both univariate and multivariate analyses indicated that high expression of biomarkers was an important risk factor for low-grade dysplasia and canceration progression (p<0.05). Meanwhile, the predictive performance of biomarkers for the risk of canceration progression was independent of clinical indicators, while gender and Helicobacter pylori infection were not independent significant risk factors (FIG. 5).

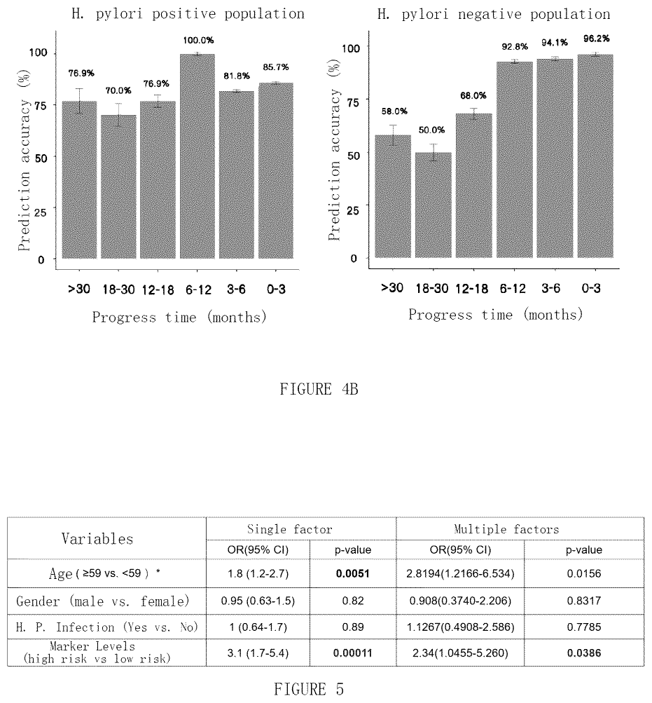

Finally, the inventors examined the expression of biomarkers at different time points during the progression of low-grade dysplasia to evaluate the association of biomarker predictive performance with the progression time window (FIG. 4A and FIG. 4B). In the overall patient population, the predictive performance of biomarkers for the risk of canceration progression was closely related to the time of progression. In the 12 months before cancer onset, the predictive accuracy significantly improves to 88%, and in the 3-6 months before cancer onset, the predictive accuracy is as high as 96%, indicating that very early gastric cancer biomarkers can effectively advance the early diagnosis window of gastric cancer by 6-12 months. Grouping based on the prevalence of Helicobacter pylori infection, it was determined that in the H. pylori negative group the biomarkers could move forward the window period by 12 months in average, with an accuracy rate of 93%, indicating that the biomarkers can further prolong the early diagnosis window for gastric cancer in the H. pylori negative population. For very early gastric cancer within 3 months, the predictive accuracy of the biomarker molecules in the overall samples reached 97%; in the Helicobacter pylori positive group, the biomarker molecules screening predicted 100% of the very early gastric cancer patients, demonstrating the diagnostic potential of the biomarkers for very early gastric cancer.

1.-9. (canceled)

10. Use of reagents for detecting the expression level of a combination of marker molecules in gastric tissue or serum samples from the tested patients in the preparation of a product for determining the risk of low-grade gastric dysplasia progress to gastric cancer and/or for determining the risk of very early gastric cancer occurrence, characterized by:
the combination of marker molecules includes one selected from the following combinations of marker molecules:
1) KLK10 and LAMC2;
2) KRT7 and LAMC2; and
3) KRT7, KLK10, and LAMC2.

11. The use according to claim 1, wherein the cells labeled in low-grade dysplasia samples by the combination of marker molecules are defined as very early gastric cancer cells, and the stage of appearance of the very early gastric cancer cells is defined as very early gastric cancer stage, and the content of very early gastric cancer cells in the test samples is quantified by evaluating the expression level of the combination of marker molecules.

12. The use according to claim 1, characterized in that:
the reagents comprise KLK10 and LAMC2 monoclonal antibodies, wherein the site sequence of the KLK10 monoclonal antibody is:
AEAALLPQNDTRLDPEAYGSPCARGSQPWQVSLFNGLSFHCAGVLVDQSW
VLTAAHCGNKPLWARVGDDHLLLLQGEQLRRTTRSVVHPKYHQGSGPILP
RRTDEHDLMLLKLARPVVLGPRVRALQLPYRCAQPGDQCQVAGWGTTAAR
RVKYNKGLTCSSITILSPKECEVFYPGVVTNNMICAGLDRGQDPCQSDSG
GPLVCDETLQGILSWGVYPCGSAQHPAVYTQICKYMSWINKVIRSN,
and the site sequence of LAMC2 monoclonal antibody is:
>sp|Q13753|22-1193
TSRREVCDCNGKSRQCIFDRELHRQTGNGFRCLNCNDNTDGIHCEKCKNG
FYRHRERDRCLPCNCNSKGSLSARCDNSGRCSCKPGVTGARCDRCLPGFH
MLTDAGCTQDQRLLDSKCDCDPAGIAGPCDAGRCVCKPAVTGERCDRCRS
GYYNLDGGNPEGCTQCFCYGHSASCRSSAEYSVHKITSTFHQDVDGWKAV
QRNGSPAKLQWSQRHQDVFSSAQRLDPVYFVAPAKFLGNQQVSYGQSLSF
DYRVDRGGRHPSAHDVILEGAGLRITAPLMPLGKTLPCGLTKTYTFRLNE
HPSNNWSPQLSYFEYRRLLRNLTALRIRATYGEYSTGYIDNVTLISARPV
SGAPAPWVEQCICPVGYKGQFCQDCASGYKRDSARLGPFGTCIPCNCQGG
GACDPDTGDCYSGDENPDIECADCPIGFYNDPHDPRSCKPCPCHNGFSCS
VMPETEEVVCNNCPPGVTGARCELCADGYFGDPFGEHGPVRPCQPCQCNN
NVDPSASGNCDRLTGRCLKCIHNTAGIYCDQCKAGYFGDPLAPNPADKCR
ACNCNPMGSEPVGCRSDGTCVCKPGFGGPNCEHGAFSCPACYNQVKIQMD
QFMQQLQRMEALISKAQGGDGVVPDTELEGRMQQAEQALQDILRDAQISE
GASRSLGLQLAKVRSQENSYQSRLDDLKMTVERVRALGSQYQNRVRDTHR
LITQMQLSLAESEASLGNTNIPASDHYVGPNGFKSLAQEATRLAESHVES
ASNMEQLTRETEDYSKQALSLVRKALHEGVGSGSGSPDGAVVQGLVEKLE
KTKSLAQQLTREATQAEIEADRSYQHSLRLLDSVSRLQGVSDQSFQVEEA
KRIKQKADSLSSLVTRHMDEFKRTQKNLGNWKEEAQQLLQNGKSGREKSD
QLLSRANLAKSRAQEALSMGNATFYEVESILKNLREFDLQVDNRKAEAEE
AMKRLSYISQKVSDASDKTQQAERALGSAAADAQRAKNGAGEALEISSEI
EQEIGSLNLEANVTADGALAMEKGLASLKSEMREVEGELERKELEFDTNM
DAVQMVITEAQKVDTRAKNAGVTIQDTLNTLDGLLHLMDQPLSVDEEGLV
LLEQKLSRAKTQINSQLRPMMSELEERARQQRGHLHLLETSIDGILADVK
NLENIRDNLPPGCYNTQALEQQ.

13. A detection reagent kit for determining the risk of progression of low-grade gastric dysplasia to gastric cancer, characterized in that:
the detection kit is used for immunohistochemical staining of a combination of molecules to obtain the expression of the combination of very early gastric cancer cell marker molecules in the tested gastric tissue samples, and/or for detecting the expression of the combination of marker molecules in blood samples through enzyme-linked immunosorbent assay,
wherein:
the combination of marker molecules includes one selected from the following combinations of marker molecules:
1) KLK10 and LAMC2;
2) KRT7 and LAMC2; and
3) KRT7, KLK10, and LAMC2.

14. The detection reagent kit according to claim 13, characterized in that:
the detection kit comprises KLK10 and LAMC2 monoclonal antibodies, wherein the site sequence of the KLK10 monoclonal antibody is:
AEAALLPQNDTRLDPEAYGSPCARGSQPWQVSLFNGLSFHCAGVLVDQSW
VLTAAHCGNKPLWARVGDDHLLLLQGEQLRRTTRSVVHPKYHQGSGPILP
RRTDEHDLMLLKLARPVVLGPRVRALQLPYRCAQPGDQCQVAGWGTTAAR
RVKYNKGLTCSSITILSPKECEVFYPGVVTNNMICAGLDRGQDPCQSDSG
GPLVCDETLQGILSWGVYPCGSAQHPAVYTQICKYMSWINKVIRSN,
and the site sequence of the monoclonal antibody against LAMC2 is:
>sp|Q13753|22-1193
TSRREVCDCNGKSRQCIFDRELHRQTGNGFRCLNCNDNTDGIHCEKCKNG
FYRHRERDRCLPCNCNSKGSLSARCDNSGRCSCKPGVTGARCDRCLPGFH
MLTDAGCTQDQRLLDSKCDCDPAGIAGPCDAGRCVCKPAVTGERCDRCRS
GYYNLDGGNPEGCTQCFCYGHSASCRSSAEYSVHKITSTFHQDVDGWKAV
QRNGSPAKLQWSQRHQDVFSSAQRLDPVYFVAPAKFLGNQQVSYGQSLSF
DYRVDRGGRHPSAHDVILEGAGLRITAPLMPLGKTLPCGLTKTYTFRLNE
HPSNNWSPQLSYFEYRRLLRNLTALRIRATYGEYSTGYIDNVTLISARPV
SGAPAPWVEQCICPVGYKGQFCQDCASGYKRDSARLGPFGTCIPCNCQGG
GACDPDTGDCYSGDENPDIECADCPIGFYNDPHDPRSCKPCPCHNGFSCS
VMPETEEVVCNNCPPGVTGARCELCADGYFGDPFGEHGPVRPCQPCQCNN
NVDPSASGNCDRLTGRCLKCIHNTAGIYCDQCKAGYFGDPLAPNPADKCR
ACNCNPMGSEPVGCRSDGTCVCKPGFGGPNCEHGAFSCPACYNQVKIQMD
QFMQQLQRMEALISKAQGGDGVVPDTELEGRMQQAEQALQDILRDAQISE
GASRSLGLQLAKVRSQENSYQSRLDDLKMTVERVRALGSQYQNRVRDTHR
LITQMQLSLAESEASLGNTNIPASDHYVGPNGFKSLAQEATRLAESHVES
ASNMEQLTRETEDYSKQALSLVRKALHEGVGSGSGSPDGAVVQGLVEKLE
KTKSLAQQLTREATQAEIEADRSYQHSLRLLDSVSRLQGVSDQSFQVEEA
KRIKQKADSLSSLVTRHMDEFKRTQKNLGNWKEEAQQLLQNGKSGREKSD
QLLSRANLAKSRAQEALSMGNATFYEVESILKNLREFDLQVDNRKAEAEE
AMKRLSYISQKVSDASDKTQQAERALGSAAADAQRAKNGAGEALEISSEI
EQEIGSLNLEANVTADGALAMEKGLASLKSEMREVEGELERKELEFDTNM
DAVQMVITEAQKVDTRAKNAGVTIQDTLNTLDGLLHLMDQPLSVDEEGLV
LLEQKLSRAKTQINSQLRPMMSELEERARQQRGHLHLLETSIDGILADVK
NLENIRDNLPPGCYNTQALEQQ.

15. A system for determining the risk of progression of low-grade gastric dysplasia to gastric cancer,
wherein:
the system determines the number of gastric cancer precancerous cells and/or very early cancer cells in the tested gastric tissue sample based on the expression of combinations of marker molecules in the tested gastric tissue sample from the tested patient, thereby determining the risk of the tested patient developing precancerous lesions and/or postoperative recurrence of gastric cancer,
the above system includes:
a detection kit (20) for immunohistochemical staining of the expression level of the combination of marker molecules (11) to obtain its expression in the tested gastric tissue sample, and/or for detecting the content of the combination of marker molecules in the blood sample of the tested patient through enzyme-linked immunosorbent assay (11);
a counting and classifying device (30) for determining classification (31) and the proportion of positive cells of the combination of marker molecules in the tested gastric tissue sample, and/or determining the overall expression level of the combination of marker molecules in the serum of the tested blood sample by calculating the average value,
wherein:
the combination of marker molecules comprises one of the following combinations of marker molecules:
1) KLK10 and LAMC2;
2) KRT7 and LAMC2; and
3) KRT7, KLK10, and LAMC2.

16. The system according to claim 15, characterized in that:
the counting and classifying device (30) are for classifying the expression levels based on the proportion to the total number of cells in the entire test tissue sample, including:
negative, proportion=0%, Grade 0,
low, proportion<5%, Grade 1,
moderate, 5%<proportion<30%, Grade 2, and
high, proportion>30%, Grade 3,
and determining the risk index for the patient's progression to gastric cancer and/or postoperative recurrence of gastric cancer; and/or for using the overall expression level of the marker molecules in the serum to determine the risk index of digestive system tumors in the tested patient.
